(19) United States
(12) Patent Application Publication
(10) Pub. No.: US 2013/0281859 A1
(43) Pub. Date: Oct. 24, 2013

(54) ULTRASOUND IMAGING SYSTEM AND METHOD

(71) Applicant: Brian A. Lause, Milwaukee, WI (US)

(72) Inventor: Brian A. Lause, Milwaukee, WI (US)

(73) Assignee: GENERAL ELECTRIC COMPANY, Schenectady, NY (US)

(21) Appl. No.: 13/453,770

(22) Filed: Apr. 23, 2012

(51) Int. Cl. A61B 8/14

(52) U.S. Cl. 600/447

(57) ABSTRACT

An ultrasound imaging system and method for generating compounded ultrasound data. The system and method includes acquiring data from two or more intersecting scan planes with a 2D array probe. At least one of the scan planes is disposed at a different angle of elevation with respect to the 2D array probe than at least one other of the scan planes. The system and method includes combining the data from the scan planes to generate compounded data.

### FIELD OF THE INVENTION

This disclosure relates generally to an ultrasound imaging system and a method for compounding ultrasound data in an elevation direction.

### BACKGROUND OF THE INVENTION

Ultrasound imaging is a technique that uses high-frequency acoustic waves to produce an image. The image is typically acquired along a series of scan lines from a transducer array. According to conventional techniques, the scan lines are typically spaced and steered to acquire data from a scan plane that may be displayed as an image. Or, data from a plurality of different scan planes may be acquired in order to acquire data of a volume.

However, when acquiring ultrasound data, some anatomical structures may be “shadowed” by objects closer to the transducer array. These anatomical structures may not be optimally imaged. Additionally, it is difficult to obtain optimal images of structures oriented in a direction that is primarily perpendicular to the transducer array since these structures reflect less acoustic energy back at the transducer array.

In addition, conventional ultrasound images typically contain speckle that degrades the image. Speckle is the result of interference of scattered echo signals reflected from anatomical structures. The speckle appears as a granular or snow-like pattern on an image. It may be difficult to identify details of small structures in an ultrasound image with speckle.

In conventional ultrasound imaging systems, it is known to combine a plurality of co-planar ultrasound data acquisitions or images into a single compounded image in order to reduce speckle, reduce shadowing, and to improve the appearance of structures that run primarily in a perpendicular direction to the transducer array. Conventional systems typically perform compounding of images sharing the same elevational plane. That is, the ultrasound imaging system combines data acquired at a first azimuth angle with data acquired at a second azimuth angle. This technique is also known as “in-plane compounding” since the scan lines that are compounded with each other are typically acquired from within the same scan plane. While in-plane compounding has proven helpful in improving image quality, there are limits to the improvements that can be made. In order for compounding to be effective, the scan lines that are combined must be acquired at significantly different angles. However, with conventional ultrasound systems, there is a limit to the maximum angle the beam may be steered in the azimuth direction. As a result, conventional ultrasound systems typically only combine three or five different beam directions within a scan plane when generating a compounded image. Additionally, since the compounding is “in-plane compounding,” the compounded data does not contain any information from intersecting or parallel scan planes. For certain anatomical structures, conventional in-plane compounding may result in images with artifacts due to shadowing.

For these and other reasons an improved method and ultrasound imaging system for generating compounded data is desired.

### BRIEF DESCRIPTION OF THE INVENTION

The above-mentioned shortcomings, disadvantages and problems are addressed herein which will be understood by reading and understanding the following specification.

In an embodiment, a method for generating compounded ultrasound data includes acquiring data from two or more intersecting scan planes with a 2D array probe, wherein at least one of the scan planes is disposed at a different angle of elevation with respect to the 2D array probe than at least one other of the scan planes. The method also includes combining the data from the scan planes to generate compounded data.

In an embodiment, a method for generating compounded ultrasound data includes acquiring first data from a first plurality of scan planes within a volume with a 2D array probe, wherein the first data is acquired along a first plurality of scan lines. The method includes acquiring second data from the first plurality of scan planes within the volume with the 2D array probe, wherein the second data is acquired along a second plurality of scan lines. Each of the second plurality of scan lines intersects at least one of the first plurality of scan lines. The method includes acquiring third data from a second plurality of scan planes within the volume with the 2D array probe. Each of the second plurality of scan planes intersects at least one of the first plurality of scan planes within the volume because the intersecting planes are disposed at different angles of elevation with respect to the 2D array probe. The method also includes combining the first data with both the second data and the third data to generate compounded data.

In another embodiment, an ultrasound imaging system includes a 2D array probe including a probe face, a display device, and a processor in electronic communication with the probe and the display device. The processor is configured to control the 2D array probe to acquire first data from a first scan plane disposed at a first angle of elevation with respect to the probe face. The processor is configured to control the 2D array probe to acquire second data from a second scan plane disposed at a second angle of elevation with respect to the probe face, wherein the first angle is different from the second angle. The processor is configured to combine the first data with the second data to generate compounded data, generate an image from the compounded data and display the image on the display device.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic representation of a 2D array probe in accordance with an embodiment;

FIG. 3 is a schematic representation of an array in accordance with an embodiment;

FIG. 4 is a flow chart shown in accordance with an embodiment;

FIG. 5 is a schematic representation of a perspective view of scan planes shown with respect to a probe face and an array in accordance with an embodiment;

FIG. 6 is a schematic representation of a perspective view of scan planes shown with respect to a probe face and an array in accordance with an embodiment;

FIG. 7 is a schematic representation of an elevational view of an array, a probe face, and a plurality of scan lines in accordance with an embodiment; and

FIG. 8 is a schematic representation of an elevation view of an array, a probe face, and a plurality of scan lines in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

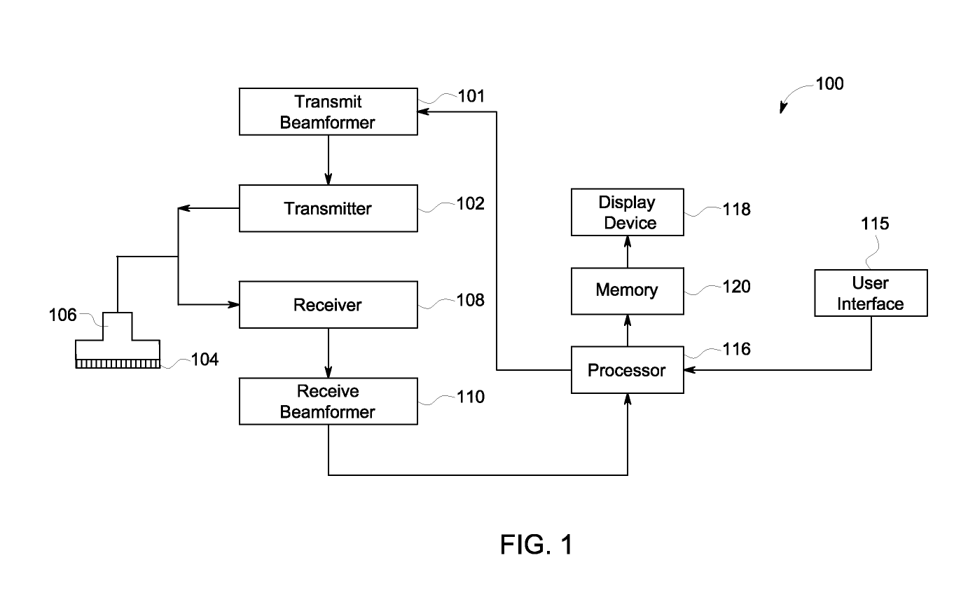

FIG. 1 is a schematic diagram of an ultrasound imaging system 100 in accordance with an embodiment. The ultrasound imaging system 100 includes a transmit beamformer 101 and a transmitter 102 that drive elements 104 within a 2D array probe 106 to emit pulsed ultrasonic signals into a body (not shown). A variety of geometries of probes and elements may be used. The pulsed ultrasonic signals are back-scattered from structures in the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. According to some embodiments, the 2D array probe 106 may contain electronic circuitry to do all or part of the transmit and/or the receive beamforming. For example, all or part of the transmit beamformer 101, the transmitter 102, the receiver 108 and the receive beamformer 110 may be situated within the 2D array probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. A user interface 115 may be used to control operation of the ultrasound imaging system 100, including, to control the input of patient data, to change a scanning or display parameter, and the like.

The ultrasound imaging system 100 also includes a processor 116 to control the transmit beamformer 101, the transmitter 102, the receiver 108 and the receive beamformer 110. The processor 116 is in electronic communication with the 2D array probe 106. The processor 116 may control the 2D array probe 106 to acquire data. The processor 116 controls which of the elements 104 are active and the shape of a beam emitted from the 2D array probe 106. The processor 116 is also in electronic communication with a display device 118, and the processor 116 may process the data into images for display on the display device 118. For purposes of this disclosure, the term “electronic communication” may be defined to include both wired and wireless connections. The processor 116 may include a central processor (CPU) according to an embodiment. According to other embodiments, the processor 116 may include other electronic components capable of carrying out processing functions, such as a digital signal processor, a field-programmable gate array (FPGA) or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment the demodulation can be carried out earlier in the processing chain. The processor 116 is adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. For example, an embodiment may acquire and display images with a real-time frame-rate of 7-20 frames/sec. However, it should be understood that the real-time frame rate may be dependent on the length of time that it takes to acquire each frame of data for display. Accordingly, when acquiring a relatively large volume of data, the real-time frame rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a frame-rate of, for example, 10 Hz to 30 Hz. Images generated from the data may be refreshed at a similar frame rate. Other embodiments may acquire and display data at different rates. For example, some embodiments may acquire data at a frame rate of less than 10 Hz or greater than 30 Hz depending on the size of the volume and the intended application. A memory 120 is included for storing processed frames of acquired data. In an exemplary embodiment, the memory 120 is of sufficient capacity to store at least several seconds worth of frames of ultrasound data. The frames of data are stored in a manner to facilitate retrieval thereof according to its order or time of acquisition. The memory 120 may comprise any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from coordinates beam space to display space coordinates. A video processor module may be provided that reads the image frames from a memory and displays the image frames in real time while a procedure is being carried out on a patient. A video processor module may store the image frames in an image memory, from which the images are read and displayed.

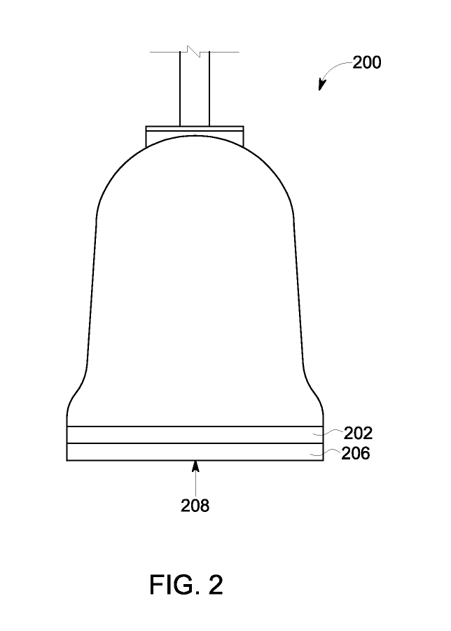

FIG. 2 is a schematic representation of a 2D array probe 200 in accordance with an embodiment. The 2D array probe 200 may be connected to the ultrasound imaging system 100 in place of 2D array probe 106. The 2D array probe 200 includes an array 202 of transducer elements and a nose piece 206. The nose piece 206 defines a probe face 208. The 2D array probe 200 may be configured to acquire data from a plurality of scan planes.

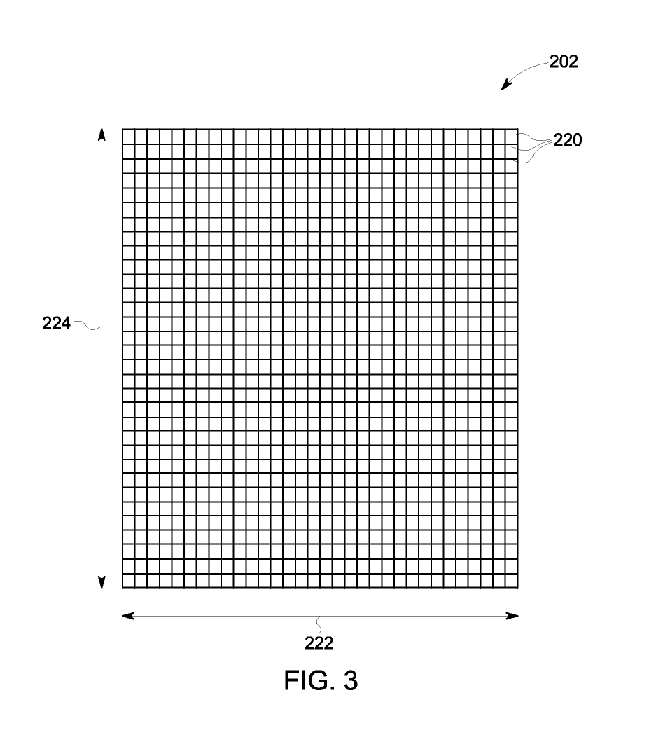

FIG. 3 is a schematic representation of the array 202 from the 2D array probe 200 (shown in FIG. 2) in accordance with an embodiment. The array 202 includes a plurality of elements 220 arranged into a 2D array, such as, for example, a grid-like pattern. Other embodiments may include 2D arrays with the elements 220 arranged in a different pattern. Additionally, the elements of other embodiments may be shaped differently than those shown in the embodiment shown in FIG. 2. The elements 220 of the probe 200 may be controlled to acquire ultrasound data, such as by acquiring data along a plurality of scan lines. The data from each of the scan lines may be combined to obtain data from a scan plane, or data from a plurality of scan planes may be acquired in order to obtain data of a volume. Data of a volume may be acquired from a plurality of scan planes that are parallel to each other or the scan planes may be arranged in a different configuration. For example, data of a volume may be acquired through the acquisition of a plurality of planes that are not parallel to each other. For instance, according to an embodiment, the scan planes may diverge from the 2D array probe in a fan-like manner. The schematic representation of the array 202 shown in FIG. 3 includes a pattern of 32 elements by 32 elements, but it should be appreciated that arrays may include any number of elements and they may be rectangular instead of square like the example shown in FIG. 3. For example, according to an exemplary embodiment, the array may include a grid of 256 elements by 132 elements. The elements 220 may be arranged in a grid that extends in both an azimuth direction 222 and an elevation direction 224. For purposes of this disclosure, the term “azimuth direction” is defined to include a direction along the transducer array 202 that is parallel to the direction of one or more scan planes, and the term “elevation direction” is defined to include the direction along the transducer array 202 that is perpendicular to the azimuth direction and the one or more scan planes. If a 2D array probe has elements arranged in a rectangular pattern, the azimuth direction will typically correspond to the direction of the array with more elements. According to the exemplary embodiment with 256 by 132 elements, the azimuth direction with typically be the direction with 256 elements while the elevation direction will be typically be the direction with 132 elements. Having additional elements in the azimuth direction may allow for the acquisition of more scan lines in each scan plane. However, it should be appreciated that scan planes may also be acquired parallel to the shorter direction of the array. In embodiments where the scan planes are parallel to the shorter direction of the array, is should be appreciated that the azimuth direction may correspond with the shorter direction of the array 202. It should also be appreciated that the array 202 does not need to be flat. The array 202 may be covered by the nose piece 206 (shown in FIG. 2) that defines the probe face 208. During use, the probe face 208 may be positioned against the patient while acquiring data. Acoustic coupling media, such as gel, may be used to aid in the transmission of ultrasound energy between the array 202 and the patient. According to embodiments, the array 202 may conform to other shapes. For example, the probe face 206 and the array 202 may be convex or concave according to other embodiments.

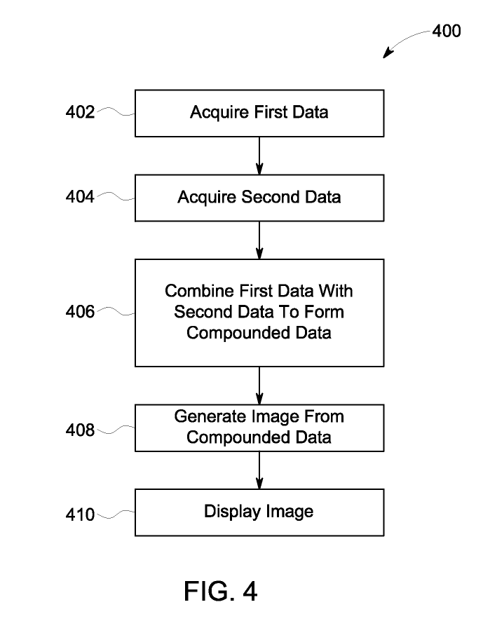

FIG. 4 is a flow chart shown in accordance with an embodiment. The individual blocks represent steps that may be performed in accordance with the method 400. Additional embodiments may perform the steps shown in a different sequence and/or additional embodiments may include additional steps not shown in FIG. 4. The technical effect of the method 400 is the generation and display of an image that is generated from compounded data.

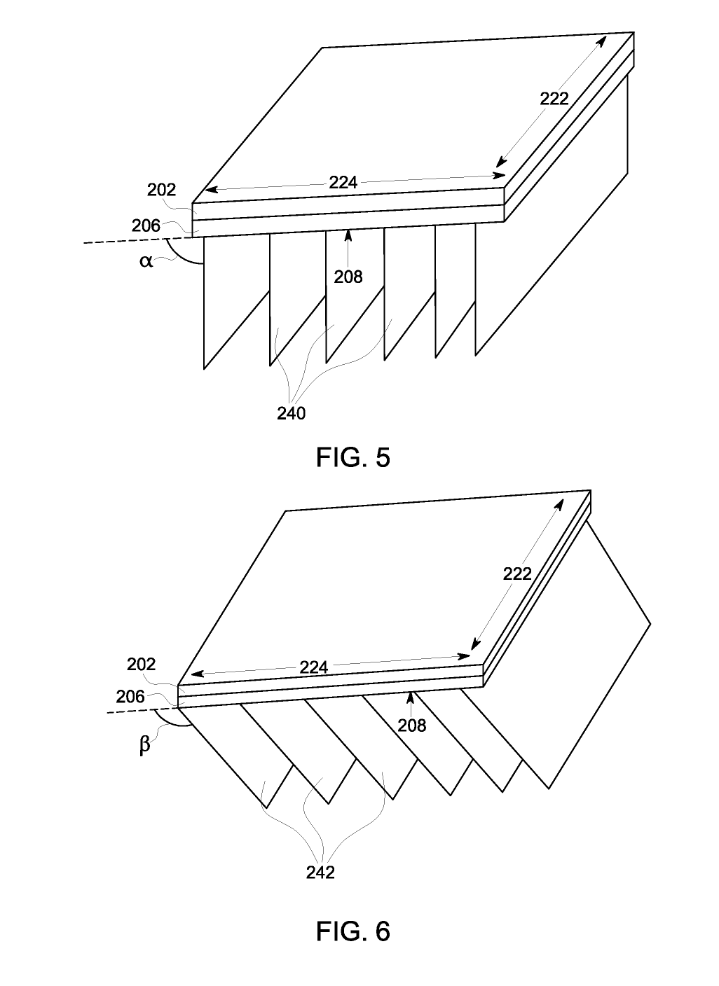

FIG. 5 is a schematic representation of a perspective view of scan planes shown with respect to a probe face and an array from a 2D array probe in accordance with an embodiment. FIG. 6 is a schematic representation of a perspective view of scan planes shown with respect to a probe face and an array from a 2D array probe in accordance with another embodiment. Common reference numbers will be used to identify identical components in FIGS. 4, 5, and 6.

Referring to FIGS. 1, 4 and 5, at step 402, the processor 116 controls the 2D array probe 106, the transmit beamformer 101, the transmitter 102, the receiver 108, and the receive beamformer 110 to acquire first data. The first data may be acquired from a first plurality of scan planes 240, which, according to an embodiment, are each disposed at an angle of elevation a with respect to the probe face 206. As discussed previously, the scan planes are disposed parallel to an azimuth direction 222, and the elevation direction 224 is perpendicular to the azimuth direction. By acquiring data from a plurality of scan planes at different locations in the elevation direction 224, the processor 116 may acquire data for a volume.

Referring to FIGS. 1, 4 and 6, at step 404, the processor 116 controls the 2D array probe 106, the transmit beamformer 101, the transmitter 102, the receiver 108, and the receive beamformer 110 to acquire second data at a second angle of elevation with respect to the probe face 206. According to an embodiment, the second data may be acquired from a second plurality of scan planes 242. The second plurality of scan planes 242 may be disposed at a different angle of elevation β with respect to the probe face 206 than the first plurality of scan planes 240. In other words, the first data and the second data are acquired along scan lines in scan planes disposed at different angles in the elevation direction. By acquiring the second data from the second plurality of scan planes 242, the processor 116 may acquire data for a second volume. Each of the second plurality of scan planes 242 may intersect with one or more of the first plurality of scan planes 240 since the first plurality of scan planes 240 are disposed at a different angle of elevation than the second plurality of scan planes 242. According to an embodiment, the first data and the second data may both include data of a common volume. However, as described hereinabove, the first data is acquired from a first angle of elevation α and the second data is acquired from a second angle of elevation β with respect to the probe face 206.

Next, at step 406, the processor 116 combines the first data with the second data to form compounded data. As described previously, the first plurality of scan planes 240 acquired at step 402 are disposed at a different angle of elevation than the second plurality of scan planes 242 acquired at step 404. Due to the different angles of elevation, the intersecting scan planes only intersect each other along a line of intersection. For the spatial position along this line of intersection, data is acquired in two different directions. However, not all of the points acquired from the scan planes 240 are in exactly the same location as the points acquired from scan planes 242. The data from the first scan planes 240 disposed at the first angle α must be combined with the data from the second scan planes 242 disposed at the second angle β with respect to the probe face 206 and mapped to a Cartesian coordinate system. Therefore, it may be necessary to use an interpolation scheme or technique in order to combine the first data with the second data. During the interpolation process values are assigned to voxels, or volume elements, in the Cartesian coordinate system based on the first data and the second data. For example, one or more values from the first data sampled from locations close to each voxel are combined with one or more values from the second data sampled from close to each voxel. According to an exemplary embodiment, the processor 116 may use a tri-linear interpolation to perform the compounding. Tri-linear interpolation is an interpolation technique that is well-known by those skilled in the art. According to other embodiments, the processor 116 may use other interpolation techniques, such as a tri-cubic interpolation to perform the compounding. Tri-linear interpolation and tri-cubic interpolation are both interpolation techniques that are well-known by those skilled in the art. It should be appreciated that other interpolation techniques may be used to combine the first data and the second data during step 406. According to other embodiments, the processor 116 may use other mathematical techniques to combine the first data with the second data to form compounded data. For example, techniques including calculating a mean, a median, a mode, a maximum, or weighted average based on the first data and the second data may also be used. It should be appreciated that both 2-d and 3-d techniques may be used.

Next, at step 408, the processor 116 generates an image from the compounded data. Since a volume of data has been compounded, the processor 116 may generate an image of any arbitrary plane through the volume. Compounding in the elevation direction, as performed at step 406, will yield a dataset capable of producing an image with improved contrast, reduced speckle and increased edge definition. Therefore, the image generated during step 408 will have improved contrast, reduced speckle and increased edge definition compared to an image generated from data that was not compounded.

Next, at step 410, the processor 116 displays the image generated during step 408 on the display device 118 (shown in FIG. 1). The image may include either a still image or a frame of a live image depending upon the embodiment.

According to other embodiments, the processor 116 may calculate a quantitative value based on the compounded data. For example, if the compounded data is of the carotid artery, then the processor 116 may calculate a quantitative value such as intima-media thickness or volume. It should be appreciated that other quantitative values, including distances, thicknesses, scores, or volumes may be calculated based on the compounded data depending upon the type of exam being performed. According to an embodiment, the quantitative value may be displayed on the display device 118 after being calculated.

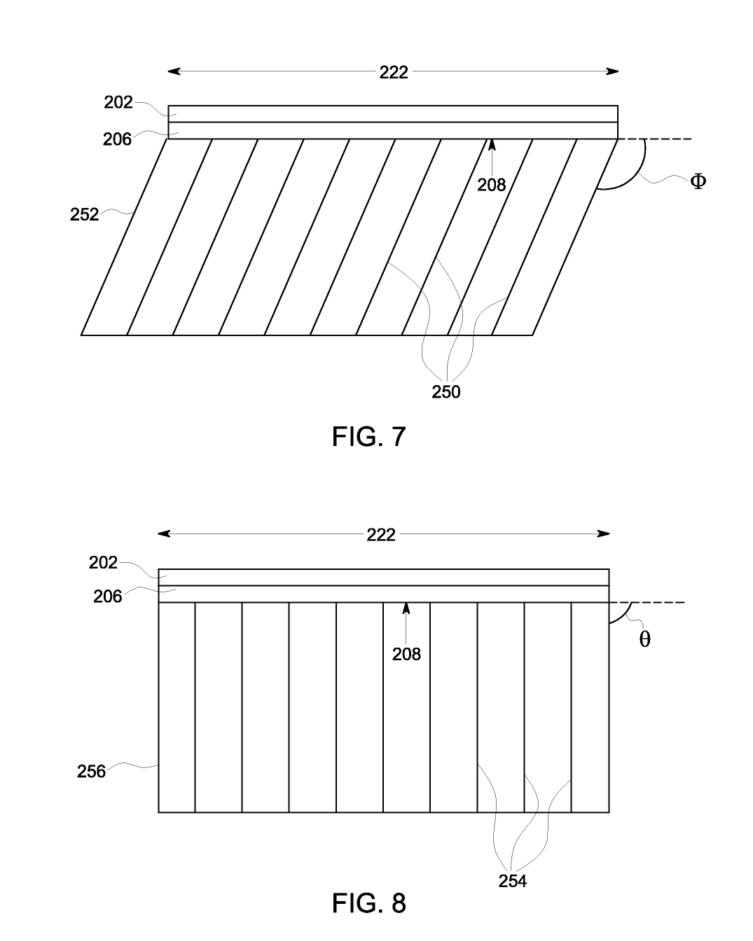

FIG. 7 is a schematic representation of an elevational view of an array, a probe face, and a plurality of scan lines in accordance with an embodiment. FIG. 8 is a schematic representation of an elevational view of an array, a probe face, and a plurality of scan lines in accordance with an embodiment. The array and the probe face shown in FIGS. 7 and 8 are the same as the array and probe face shown in FIGS. 5 and 6. Common reference numbers will be used to identify identical components in FIGS. 5, 6, 7, and 8.

According to other embodiments, the method 400 may be modified so that the processor 116 (shown in FIG. 1) controls the acquisition of third data with the 2D array probe 200 (shown in FIG. 2). According to an embodiment, the third data may include data from the first plurality of scan planes. However, the third data may be acquired along scans lines disposed at different angles of azimuth than the scan lines used to acquire the first data. For example, referring to FIG. 7, the processor 116 may control the 2D array probe to acquire a first plurality of scan lines 250 at a first angle of azimuth Φ. It should be noted that the first plurality of scan lines 250 are all in the same scan plane 252 and that the first angle of azimuth Φ is determined with respect to the probe face 208. Referring to FIG. 8, the processor may then control the 2D array probe 200 to acquire a second plurality of scan lines 254 disposed at a second angle of azimuth θ with respect to the probe face 208. The second plurality of scan lines 254 are all disposed in the same scan plane 256. The first angle of azimuth Φ (shown in FIG. 7) is different from the second angle of azimuth θ (shown in FIG. 8). Referring to both FIGS. 7 and 8, the scan plane 252 and the scan plane 256 are co-planar, meaning they both define the same plane. As such, the first plurality of scan lines 250 may be compounded with the second plurality of scan lines 254. FIGS. 7 and 8 only show one representative scan plane within a volume. It should be appreciated that additional scan planes may be acquired and compounded at multiple angles of azimuth. Then, the processor 116 may combine the first data with both the second data and the third data to generate the compounded data. According to this exemplary embodiment, the first data is acquired along scan lines at a different angle of azimuth than the third data. Additionally, the first data and the second data are acquired from scan planes, and hence along scan lines, that are disposed at different angles of elevation with respect to the probe face 208.

According to the exemplary embodiment, a volume, or volume of interest, may be included in each of the three datasets. That is, a common volume may be included in the first data, the second data, and the third data. However, each voxel is assigned a value based on data acquired along at least three unique scan lines. When combined, the compounded data is compounded in both an elevation direction and an azimuth direction. For purposes of this disclosure, the term “compounded in the elevation direction” is defined to include combining data acquired at two or more different angles of elevation and the term “compounded in the azimuth direction” is defined to include combining data acquired at two or more different angles of azimuth.

Generating compounded data that is compounded in both an elevation direction and an azimuth direction is beneficial because it facilitates higher quality data. For example, as described previously, there are limits to the amount of beam steering that may be applied to the beams when performing in-plane compounding. Due to this restriction, there are a limited number of scan lines that may be acquired in a given scan plane for the purposes of compounding. By adding elevational compounding, that is generating compounded data based on scan lines or scan planes acquired at two or more different angles of elevation, it is possible to acquire data along additional unique scan lines. Combining data from more unique scan lines, by techniques such as interpolation, will result in higher image quality. Fully steerable probes, such as the 2D array probe 200 (shown in FIG. 2) allow for the acquisition scan lines from a wide range of angles in both the elevation and azimuth directions.

Compounded data that is compounded in two or more directions, such as in the azimuth and elevation directions, allows each pixel or voxel of the compounded data, or any image generated from the compounded data, to have higher image quality than images generated from conventional compounded data. Specifically, compounded data acquired in accordance with embodiments described in this disclosure will have reduced speckle, reduced shadowing and better visualization of objection oriented in a generally perpendicular direction to the probe. Since speckle in caused by interference from reflected ultrasound waves, images generated from compounded data in accordance with the embodiments described will have less speckle because the data from two or more different directions are not coherent and will be averaged together. Using data that is compounded from more unique directions will result in a stronger coherence in true anatomical signal and weaker coherence in speckle signal and, hence, less speckle. If the patient's anatomy being imaged contains elongated structures that run in a direction substantially within a scan plane, then compounding in the elevation, or out-of-plane direction will potentially reduce the shadowing of anatomy below the elongated structures. Additionally, by combining data acquired at different angles of elevation, the compounded data will be more likely to contain a strong signal from elongated structures that are substantially perpendicular to the array.

It should be appreciated, that according to other embodiments, additional data may be acquired. For example, data may be acquired from more than two different angles of elevation and data may be acquired from more than two different angles of azimuth.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method for generating compounded ultrasound data comprising:
acquiring data from two or more intersecting scan planes with a 2D array probe, wherein at least one of the scan planes is disposed at a different angle of elevation with respect to the 2D array probe than at least one other of the scan planes; and
combining the data from the scan planes to generate compounded data.

2. The method of claim 1, further comprising generating an image from the compounded data.

3. The method of claim 2, further comprising displaying the image.

4. The method of claim 1, further comprising calculating a quantitative value based on the compounded data.

5. The method of claim 4, further comprising displaying the quantitative value.

6. The method of claim 1, wherein said acquiring data from the two or more intersecting scan planes comprises acquiring first data from a first plurality of scan planes within a volume and acquiring second data from a second plurality of scan plane within the volume, wherein each of the second plurality of scan planes intersects at least one of the first plurality of scan planes within the volume because the intersecting scan planes are disposed at different angles of elevation with respect to the probe face.

7. The method of claim 6, wherein said combining the data comprises combining all of the first data within the volume with all of the second data within the volume.

8. The method of claim 6, wherein said combining the data comprises interpolating the first data and the second data.

9. The method of claim 8, wherein said interpolating comprises using either a tri-linear interpolation or a tri-cubic interpolation.

10. A method for generating compounded ultrasound data comprising:
acquiring first data from a first plurality of scan planes within a volume with a 2D array probe, wherein the first data is acquired along a first plurality of scan lines;
acquiring second data from the first plurality of scan planes within the volume with the 2D array probe, wherein the second data is acquired along a second plurality of scan lines, wherein each of the second plurality of scan lines intersects at least one of the first plurality of scan lines;
acquiring third data from a second plurality of scan planes within the volume with the 2D array probe, wherein each of the second plurality of scan planes intersects at least one of the first plurality of scan planes within the volume because the intersecting scan planes are disposed at different angles of elevation with respect to the 2D array probe; and
combining the first data with both the second data and the third data to generate compounded data.

11. The method of claim 10, further comprising generating an image from the compounded data.

12. The method of claim 10, further comprising displaying the image.

13. The method of claim 10, wherein said combining the first data with both the second data and the third data comprises interpolating the first data, the second data, and the third data.

14. The method of claim 10, wherein said combining the first data with both the second data and the third data comprises one of calculating a mean, calculating a mode, and calculating a maximum.

15. An ultrasound imaging system comprising:
a 2D array probe including a probe face;
a display device; and
a processor in electronic communication with the probe and the display device, wherein the processor is configured to:
control the 2D array probe to acquire first data from a first scan plane disposed at a first angle of elevation with respect to the probe face;
control the 2D array probe to acquire second data from a second scan plane disposed at a second angle of elevation with respect to the probe face, wherein the first angle is different from the second angle;
combine the first data with the second data to generate compounded data;
generate an image from the compounded data; and
display the image on the display device.

16. The ultrasound imaging system of claim 15, wherein the processor is further configured to combine the first data with the second data by interpolating the first data and the second data using either a tri-linear or a tri-cubic interpolation.

17. The ultrasound imaging system of claim 15, further comprising a software beamformer connected to the 2D array probe and the processor

18. The ultrasound imaging system of claim 15, wherein the processor is further configured to control the 2D array probe to acquire third data from the first scan plane, wherein the third data is acquired at a different angle in the azimuth direction than the first data.

19. The ultrasound imaging system of claim 18, wherein the processor is further configured to combine the third data with both the first data and the second data to generate the compounded data.

20. The ultrasound imaging system of claim 15, wherein the processor is further configured to combine the first data with the second data along a line of intersection between the first scan plane and the second scan plane.
